(19) United States
(12) Patent Application Publication
(10) Pub. No.: US 2006/0199882 A1
(43) Pub. Date: Sep. 7, 2006

(54) Heterocyclic dyes based on benzodipyrroles

(76) Inventors: Heino Heckmann, Liederbach (DE); Andre Weiss, Augsburg (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 10/550,995

(22) PCT Filed: Feb. 24, 2004
(86) PCT No.: PCT/EP04/01799

§ 371 Date: Sep. 26, 2005

(30) Foreign Application Priority Data
Mar. 27, 2003 (DE) 103 13 701.7

(51) Int. Cl. C08K 5/34; C07D 487/02

(52) U.S. Cl. 524/90; 544/310; 544/251; 546/155; 548/364.4; 548/428

(57) ABSTRACT

The invention relates to a compound of general formula (I), in which A stands for the bivalent group of a cyclic compound of general formula (II), in which B is an alicyclic or heterocyclic group and A¹, A² and A³ are identical or different and are defined as per A or represent ═NR, whereby R represents hydrogen, unsubstituted halogen or halogen substituted by 1, 2, 3 or 4 groups, R⁰, OR⁰, SR⁰, NH₂, NH₂, NHR⁰, NR⁰₂, NO₂, COOH, COOR⁰, CONH₂, CONHR⁰, CONR⁰₂, CN, SO₃H, SO₂ (OR⁰), SO₂R⁰, or a phenyl substituted by a 5- to 7-membered heteroaromatic group with 1, 2 or 3 heteroatoms from the group N, O and S; a 5- to 7-membered heteroaromatic group with 1, 2 or 3 heteroatoms from the group N, O and S; NH₂, NHR⁰, NR⁰₂, NHCONH₂ or NHCONHR⁰, whereby R⁰ stands for C₁-C₁₈ alkyl or C₆-C₂₄ aryl.

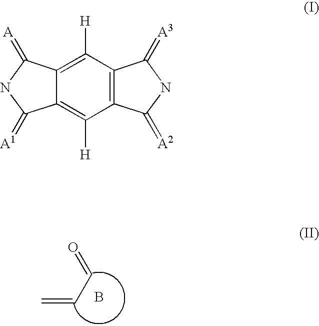

The present invention relates to novel organic pigments and dyes.

There is a constant commercial demand for colorants having novel hues possessing high migration and light fastnesses, good thermal stabilities and high coloring intensity and also additionally, in the case of pigments, high solvent fastnesses.

Saltlike compounds comprising the dianion of 2,2′,2″,2′″-benzo[1,2-c:4,5-c′]dipyrrole-1,3,5,7(2H,6H)-tetraylidenetetrakispropanedinitrile are described in Angew. Chem. 1987, 99(10), pages 1068-1070. These compounds and their starting materials are obtained in a costly and inconvenient sequence of syntheses. Their low fastnesses disqualify these compounds from use as colorants.

It is an object of the present invention to provide novel colorants for dyeing or pigmenting organic or inorganic material of high or low molecular weight, in particular high molecular weight organic material, which are based on readily obtainable intermediates.

We have found that this object is achieved, surprisingly, by compounds of the formula (I).

One aspect of the present invention accordingly provides compounds of the general formula (I)

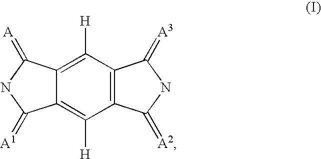

wherein A represents the divalent radical of a cyclic compound of the general formula (II)

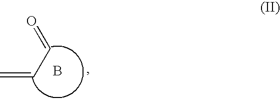

where B is an alicyclic or heterocyclic group, and

A¹, A² and A³ are the same or different and have the meaning of A or represent ═NR, where R is hydrogen, unsubstituted phenyl, phenyl substituted by 1, 2, 3 or 4 radicals selected from the group consisting of halogen, R⁰, OR⁰, SR⁰, NH₂, NHR⁰, NR⁰₂, NO₂, COOH, COOR⁰, CONH₂, CONHR⁰, CONR⁰₂, CN, SO₃H, SO₂(OR⁰), SO₂R⁰, or by a 5- to 7-membered heteroaromatic radical having 1, 2 or 3 heteroatoms from the group consisting of N, O and S; a 5- to 7-membered heteroaromatic radical having 1, 2 or 3 heteroatoms from the group consisting of N, O and S; and NH₂, NHR⁰, NR⁰₂, NHCONH₂ or NHCONHR⁰, where R⁰ is C₁-C₁₈-alkyl or C₆-C₂₄-aryl.

Preference is given to compounds of the formula (I) wherein one of A¹, A² and A³, more preferably A² or A³, has the meaning of A.

Preference is further given to compounds of the formula (I) wherein two of A¹, A² and A³, more preferably A¹ and A², have the meaning of A.

Preference is further given to compounds of the formula (I) wherein A¹, A² and A³ have the meaning of A.

Particular preference is given to compounds of the general formula (I) wherein A represents the divalent radical of a cyclic compound of the general formula (III)

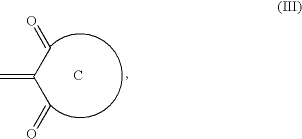

where C is an alicyclic or heterocyclic group.

Very particular preference is given to compounds of the general formula (I) wherein A is a divalent radical of the formulae (a) to (g)

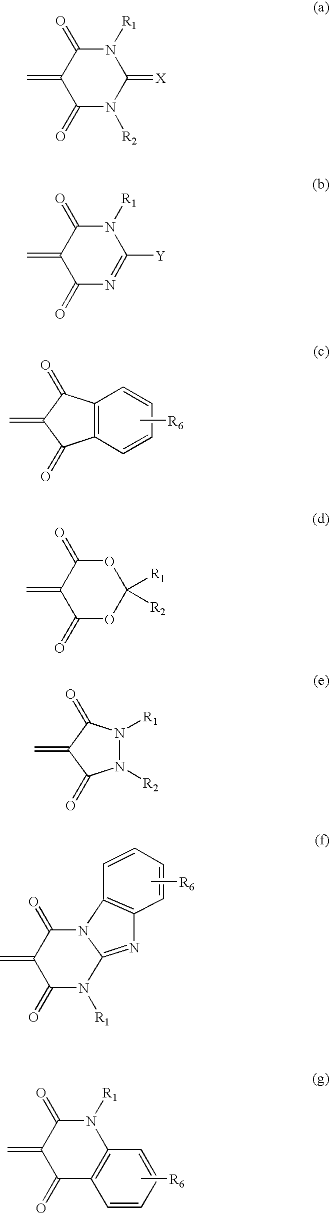

where R₁ and R₂ independently represent hydrogen, C₁-C₂₅-alkyl, C₅-C₁₂-cycloalkyl, C₆-C₂₄-aryl, C₁-C₂₅-alkyl-(C₆-C₁₀-aryl), a 5- to 7-membered heteroaromatic radical having 1, 2 or 3 heteroatoms from the group consisting of N, O and S, —(CH₂)ₙ—COR₃ or —(CH₂)ₘ—OR₄,

where R₃ is hydroxyl, amino or unsubstituted or singly or multiply, for example 1-, 2-, 3- or 4-tuply, hydroxyl- or amino-substituted C₁-C₂₅-alkoxy, C₁-C₂₅-alkylamino, di(C₁-C₂₅-alkyl)amino, C₆-C₂₄-arylamino, di(C₆-C₂₄-aryl)amino, C₁-C₂₅-alkyl-(C₆-C₁₀-aryl)amino or C₂-C₂₄-alkenyloxy;

R₄ is hydrogen or —CO—(C₁-C₂₅-alkyl), and

n and m are independently an integer from 0 to 6 and preferably from 1 to 4, and where a C—C unit in R₁, R₂, R₃ and R₄ may also be replaced by an ether unit C—O—C;

X is ═O, ═S or ═NR₅, where R₅ has the same meaning as R₁ or R₂;

Y is hydrogen, R₂, OR₂, SR₂, NHCN or NR₂R₅; and R₆ is hydrogen, halogen, CN, R₂, OR₂, SR₂, NR₂R₅, NO₂, SO₂(OR₂), SO₂R₂, SO₂NR₂R₅ or PO₂(OR₂).

More preferably R₁ and R₂ are hydrogen, C₁-C₁₈-alkyl, C₅-C₆-cycloalkyl, C₆-C₁₀-aryl, benzyl, pyridyl, pyrryl, thienyl, imidazolyl, oxazolyl, thiazolyl, pyrimidyl, hydroxycarbonyl-C₀-C₆-alkyl, C₁-C₁₈-alkoxycarbonyl-C₀-C₆-alkyl, aminocarbonyl-C₀-C₆-alkyl, C₁-C₁₈-alkylaminocarbonyl-C₀-C₆-alkyl, C₆-C₁₀-arylaminocarbonyl-C₀-C₆-alkyl, di(C₁-C₁₈-alkyl)aminocarbonyl-C₀-C₆-alkyl, C₁-C₁₈-alkyl-C₆-C₁₀-arylaminocarbonyl-C₀-C₆-alkyl or di(C₆-C₁₀-aryl)aminocarbonyl-C₀-C₆-alkyl.

More preferably R₃ is hydroxyl, C₁-C₁₈-alkoxy, C₁-C₁₈-alkylamino, di(C₁-C₁₈-alkyl)amino, benzylamino, C₆-C₁₀-arylamino, di(C₆-C₁₀-aryl)amino or (C₂-C₁₈)-alkenyloxy.

More preferably R₆ is hydrogen, Cl, Br, C₁-C₁₈-alkyl, C₅-C₆-cycloalkyl, benzyl, C₆-C₁₀-aryl, pyridyl, pyrryl, thienyl, imidazolyl, oxazolyl, thiazolyl, pyrimidyl, C₁-C₁₈-alkoxy, C₆-C₁₀-aryloxy, C₁-C₁₈-alkylthio, C₆-C₁₀-arylthio, C₁-C₁₈-alkylamino, C₆-C₁₀-arylamino, di(C₁-C₁₈-alkyl)amino, C₁-C₁₈-alkyl-C₆-C₁₀-arylamino, di(C₆-C₁₀-aryl)amino, SO₃H, C₁-C₁₈-alkoxysulfonyl, C₁-C₁₈-alkylsulfonyl or di(C₁-C₁₈-alkyl)aminosulfonyl.

The present invention further provides a process for preparing compounds of the general formula (I) by reacting 1,2,4,5-tetracyanobenzene with at least 2 equivalents of ammonia and/or alkoxides MOR₇, where M is sodium or potassium, R₇ is C₁-C₁₈-alkyl or —(CH₂)ₘ—OH and m is an integer from 1 to 6, and a C—C unit may also be replaced by an ether unit C—O—C, for example sodium methoxide, sodium ethoxide, sodium amylate, potassium methoxide or potassium tert-butoxide, to form tetra-, tri-, di- or monoimino-substituted benzodipyrroles which may bear 0 to 5 alkoxy substituents, exemplarily represented by compounds of the formulae (IV), (V) or (VI)

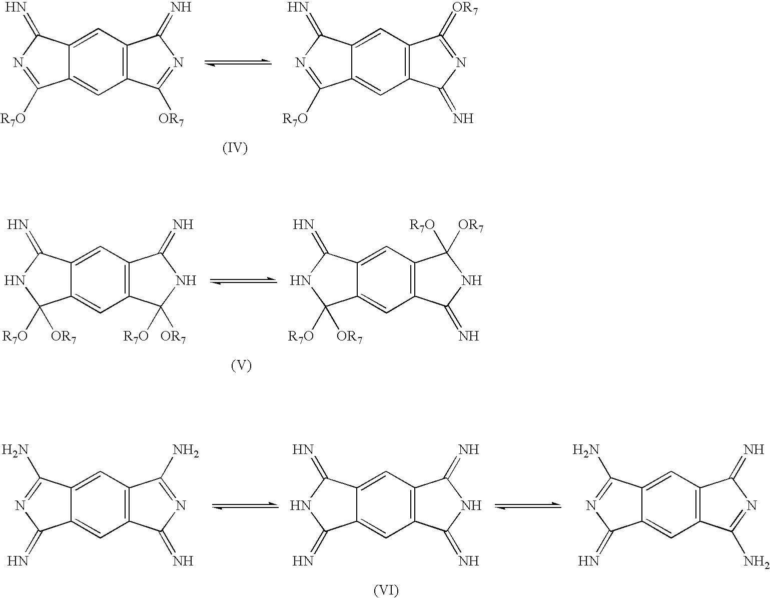

in a solvent or solvent mixture under basic to neutral conditions, at a temperature in the range from −20 to 120° C., preferably in the range from 0 to 100° C. and more preferably in the range from 20 to 80° C.,

which are subsequently, after intervening isolation or without being interveningly isolated, reacted in a solvent or solvent mixture under neutral to acidic conditions, preferably in the presence of an organic acid, for example formic acid, acetic acid and propionic acid, or of an inorganic acid, for example sulfuric acid, hydrochloric acid and phosphoric acid, and advantageously at a temperature in the range from 10 to 250° C., especially in the range from 20 to 200° C. and more preferably in the range from 30 to 150° C., with at least 1 equivalent of a cyclic compound of the formula (VII)

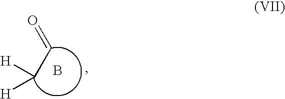

and if appropriate not more than 3 equivalents of H₂NR, where R is as defined above.

Advantageously, the reaction of the present invention is carried out in the presence of a solvent such as methanol, ethanol, glycols, dichloromethane, chloroform, dimethylformamide, N-methylpyrrolidone, toluene, mono-, di- or trichlorobenzene or a mixture thereof.

Compounds of the general formula (I) according to the present invention are used for dyeing or pigmenting organic or inorganic, high or low molecular weight, in particular high molecular weight organic, materials.

Depending on the identity of their substituents and of the high molecular weight organic material to be colored, the compounds of the present invention can be used as polymer-soluble dyes or as pigments. In the latter case it is advantageous to convert the as-synthesized products (crude pigments) by aftertreatment in organic solvents and at elevated temperatures, for example at 60 to 200° C., in particular at 70 to 150° C. and preferably at 75 to 100° C., into a finely disperse form often having further improved pigmentary properties. The aftertreatment is preferably combined with a grinding or kneading operation.

The colorants of the invention are outstandingly suitable for coloring high molecular weight materials, which may be organic or inorganic in nature, and are plastics and/or natural materials. These may be, for example, natural resins, drying oils, rubber or casein. Alternatively they may be modified natural materials, such as chlorinated rubber, oil-modified alkyd resins, viscose, cellulose derivatives, such as cellulose esters or cellulose ethers, and, in particular, synthetic organic polymers (plastics), which may be obtained by addition polymerization, polycondensation or polyaddition. From the class of the plastics prepared by addition polymerization mention may be made in particular of the following: polyolefins, such as polyethylene, polypropylene, polyisobutylene, and substituted polyolefins such as polystyrene, polyvinyl chloride, polyvinylidene chloride, polyvinylacetals, polyacrylonitrile, polyacrylic acid, polymethacrylic acid, polyacrylic esters and polymethacrylic esters or polybutadiene, for example, and also copolymers thereof.

From the class of the plastics prepared by polyaddition and polycondensation mention may be made of the following: polyesters, polyamides, polyimides, polycarbonates, polyurethanes, polyethers, polyacetals, and also the condensation products of formaldehyde with phenols (phenolic resins) and the condensation products of formaldehyde with urea, thiourea, and melamine (amino resins). The materials in question may also be silicones or silicone resins.

High molecular weight materials of this kind can be present individually or in mixtures in the form of plastic masses or melts or in the form of spinning solutions. They may also be present in the form of their monomers or in the polymerized state, in dissolved form, as film formers or binders for paints or printing inks, such as linseed oil varnish, nitrocellulose, alkyd resins, melamine resins and formaldehyde resins or acrylic resins.

The compounds of the invention are suitable, accordingly, as colorants in oil-based or water-based paints, in coating materials of various kinds, camouflage paints, for spin coloring, for the mass coloring or pigmenting of plastics, in printing inks for the graphics industry, such as, for example, in paper, textile or decorative printing, and in the mass coloring of paper, for preparing inks, water-based or non-water-based ink-jet inks, microemulsion inks, and inks which operate in accordance with the hot-melt process.

The compounds of the invention are also suitable for use as colorants in electrophotographic toners and developers, such as, for example, one- or two-component powder toners (also called one- or two-component developers), magnetic toners, liquid toners, latex toners, polymerization toners and specialty toners.

Typical toner binders are addition polymerization resins, polyaddition resins and polycondensation resins, such as styrene, styrene-acrylate, styrene-butadiene, acrylate, polyester, phenol-epoxy resins, polysulfones, polyurethanes, individually or in combination, and also polyethylene and polypropylene, which may include further ingredients, such as charge control agents, waxes or flow assistants, or are modified subsequently with these additions.

The compounds of the invention are additionally suitable for use as colorants in powders and powder coating materials, particularly in triboelectrically or electrokinetically sprayable powder coating materials which are employed to coat the surfaces of articles made, for example, of metal, wood, plastic, glass, ceramic, concrete, textile material, paper or rubber.

Typical powder coating resins used include epoxy resins, carboxyl- and hydroxyl-containing polyester resins, polyurethane resins and acrylic resins, together with customary hardeners. Resin combinations are also used. Thus, for example, epoxy resins are frequently used in combination with carboxyl- and hydroxyl-containing polyester resins. Typical hardener components (depending on the resin system) are, for example, acid anhydrides, imidazoles and also dicyandiamide and their derivatives, blocked isocyanates, bisacylurethanes, phenolic resins, melamine resins, triglycidyl isocyanurates, oxazolines and dicarboxylic acids.

Additionally the compounds of the invention are suitable for use as colorants in inks, preferably ink-jet inks, for example water-based or nonwater-based ink-jet inks, microemulsion inks and also in those inks which operate in accordance with the hot-melt process.

Ink-jet inks contain in general a total of 0.5% to 15%, preferably 1.5% to 8%, by weight (calculated on a dry basis) of one or more of the compounds of the invention.

Microemulsion inks are based on organic solvents, water and, if desired, an additional hydrotropic substance (interface mediator).

Microemulsion inks contain 0.5% to 15%, preferably 1.5% to 8%, by weight of one or more of the compounds of the invention, 5% to 99% by weight of water and 0.5% to 94.5% by weight of organic solvent and/or hydrotropic compound.

Solvent-based ink-jet inks contain preferably 0.5% to 15% by weight of one or more of the compounds of the invention, 85% to 99.5% by weight of organic solvent and/or hydrotropic compounds.

Hot-melt inks are based mostly on waxes, fatty acids, fatty alcohols or sulfonamides which are solid at room temperature and liquefy on heating, the preferred melting range being situated between about 60° C. and about 140° C. Hot-melt ink-jet inks are composed, for example, essentially of 20% to 90% by weight of wax and 1% to 10% by weight of one or more of the compounds of the invention. Additionally there may be 0% to 20% by weight of an additional polymer (as “dye dissolver”), 0% to 5% by weight of dispersing assistants, 0% to 20% by weight of viscosity modifiers, 0% to 20% by weight of plasticizers, 0% to 10% by weight of tack additive, 0% to 10% by weight of transparency stabilizer (which prevents, for example, the waxes crystallizing) and 0% to 2% by weight of antioxidant.

The compounds of the invention are also suitable, moreover, for use as colorants for color filters, both for additive and for subtractive color generation, and also as colorants for electronic inks (“e-inks”) or electronic paper (“e-paper”).

In the production of what are called color filters, both reflective and transparent color filters, pigments in the form of a paste or as pigmented photoresists in suitable binders (acrylates, acrylic esters, polyimides, polyvinyl alcohols, epoxides, polyesters, melamines, gelatin, caseins) are applied to the respective LCD components (e.g. TFT-LCD=thin film transistor liquid crystal displays or, e.g., ((S) TN-LCD=(super) twisted nematic-LCD). Besides high thermal stability, high pigment cleanness is a prerequisite for a stable paste or a pigmented photoresist. Furthermore, the pigmented color filters may also be applied by ink-jet printing processes or other suitable printing processes.

The present invention provides, moreover, for the use of the colorants of the invention in optical layers for optical data storage, preferably for optical data storage where a laser is used to write the data. The solubility of the colorants in the application medium, which is needed for this application, can be adjusted by means of the identity and number of the substituents.

The compounds of the invention are, furthermore, suitable for use as colorants in cosmetics, for coloring seed, and for coloring mineral oils, lubricating greases, and waxes.

Depending on the nature of the substituents of the compounds of the invention the colorations obtained are distinguished by good heat fastness, light fastness and weather fastness, by chemical resistance and by the very good applications properties, e.g., crystallization fastness and dispersion fastness, and in particular by their migration fastness, bleed fastness, fastness to overcoating, and solvent fastness. The compounds used as polymer-soluble dyes naturally have only little, or restricted, solvent fastness.

The invention additionally provides a composition comprising an organic or inorganic, high or low molecular weight material, particularly high molecular weight organic material, and at least one compound of the invention in a coloristically effective amount, generally in the range from 0.005% to 70% by weight, in particular from 0.01% to 10% by weight, based on the organic or inorganic material.

### EXAMPLES

### Preparation of benzo[1,2-c:4,5-c′]dipyrrole-1,3,5,7(2H,6H)-tetraimine

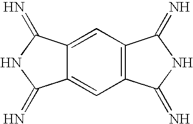

Ammonia gas is introduced at 55-60° C. into a mixture of 20.0 g of 1,2,4,5-tetracyanobenzene in 500 ml of ethylene glycol until the starting material is completely converted (verification by thin layer chromatography). The mixture is cooled down to 20° C. and stirred for a further 4 h. The product is filtered off with suction and washed with ethylene glycol and acetone. Drying at 20° C. under reduced pressure leaves 23.4 g (98% of theory) of a beige powder of the above formula.

MS (m/e): 213 [M+H]⁺

### Example 1

### 5,5′,5″,5′″-Benzo[1,2-c:4,5-c′]dipyrrole-1,3,5,7(2H,6H)-tetraylidenetetrakis-1,3-dimethyl-2,4,6-trioxotetrahydropyrimidine

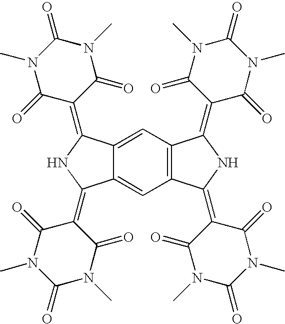

a) 5.3 g of benzo[1,2-c:4,5-c′]dipyrrole-1,3,5,7(2H,6H)-tetraimine and 23.4 g of 1,3-dimethylbarbituric acid are placed in 225 ml of glacial acetic acid and first stirred at room temperature for 30 min and then heated at the boil for 6 hours. The suspension is cooled down to room temperature, filtered off, washed in succession with glacial acetic acid, methanol and hot water (about 90° C.) and subsequently dried at 60° C. The yield is 13.2 g (69% of theory) of an orange powder of the above formula.

Melting point: >300° C.

MALDI (m/e): 768 [M−H]⁻

H NMR (D₂SO₄): 9.34 (s, 2H), 2.73 (s, 24H)

b) A suspension of 11.3 g of 1,2,4,5-tetracyanobenzene and 3.0 g of sodium methoxide solution (30% in methanol) in 340 ml of methanol is stirred at room temperature for 16 hours. 6.8 g of glacial acetic acid and 43.4 g of 1,3-dimethylbarbituric acid are added and the reaction mixture is subsequently heated at room temperature for 24 hours and then at the boil for 6 hours. The suspension is cooled down to room temperature and filtered. The crude product is purified by stirring in 250 ml of glacial acetic acid at the boil for 15 min, hot filtration with suction, successive washing with glacial acetic acid and hot water (about 90° C.) and subsequent drying at 60° C. The yield is 43.2 g (89% of theory) of an orange powder of the above formula.

Melting point: >300° C.

MALDI (m/e): 768 [M−H]⁻

### Example 2

### Reaction of benzo[1,2-c:4,5-c′]dipyrrole-1,3,5,7(2H,6H)-tetraimine with aniline and 1,3-dimethylbarbituric acid

5.3 g of benzo[1,2-c:4,5-c′]dipyrrole-1,3,5,7(2H,6H)-tetraimine and 7.0 g of aniline are stirred in 150 ml of glacial acetic acid at room temperature for 24 hours, 7.8 g of 1,3-dimethylbarbituric acid are added, and the mixture is subsequently heated at the boil for 6 hours. The suspension is cooled down to room temperature, filtered off, washed in succession with glacial acetic acid, methanol and hot water (about 90° C.) and subsequently dried at 60° C. The yield is 9.26 g of an orange powder of a mixture of the following formulae:

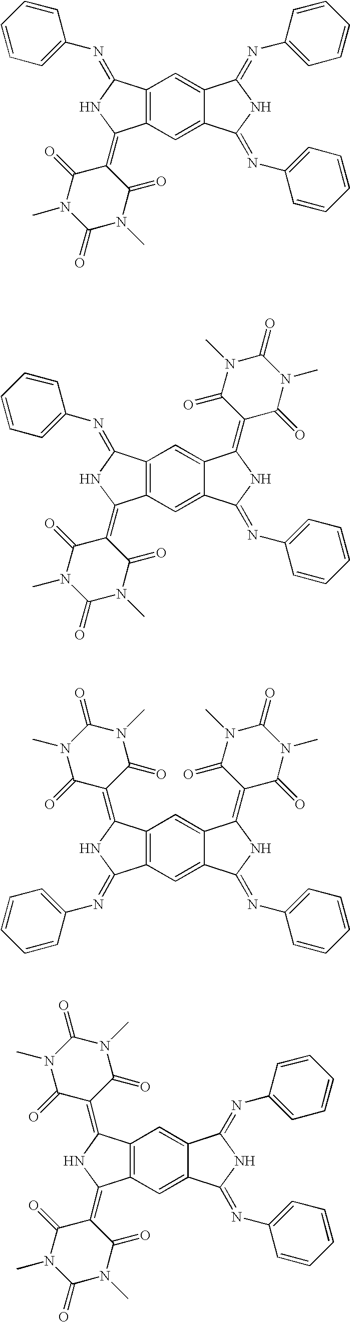

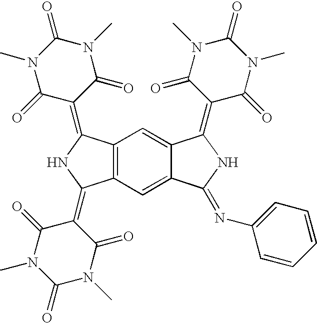

Melting point: >300° C.

MALDI (m/e): 644, 707, 770 [M+H]⁺

### Example 3

### 5,5′,5″,5′″-Benzo[1,2-c:4,5-c′]dipyrrole-1,3,5,7(2H,6H)-tetraylidenetetrakis-1,3-bis(2-ethylhexyl)-2,4,6-trioxo-tetrahydropyrimidine

1.06 g of benzo[1,2-c:4,5-c′]dipyrrole-1,3,5,7(2H,6H)-tetraimine and 8.48 g of 1,3-bis(2-ethylhexyl)barbituric acid are stirred in a mixture of 90 ml of methanol and 30 ml of glacial acetic acid at room temperature for 24 hours. The suspension is subsequently filtered off, washed with methanol and then hot water (about 90° C.) and dried at 60° C. Purification is effected by column chromatography over silica gel using a mobile phase mixture of toluene and hexane. The yield is 1.20 g (15% of theory) of a red powder of the following formula:

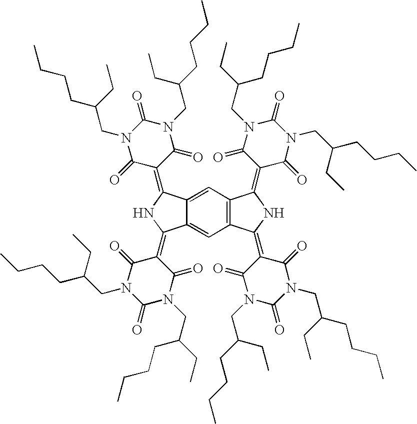

Melting point: 113-115° C.

MS (m/e): 1554 [M−H]⁻

### Example 4

### 5,5′,5″,5′″-Benzo[1,2-c:4,5-c′]dipyrrole-1,3,5,7(2H,6H)-tetraylidenetetrakis-1,3-dibutyl-2,4,6-trioxotetrahydropyrimidine

1.06 g of benzo[1,2-c:4,5-c′]dipyrrole-1,3,5,7(2H,6H)-tetraimine and 6.01 g of 1,3-dibutylbarbituric acid are stirred in a mixture of 10 ml of glacial acetic acid and 40 ml of toluene at room temperature for 24 h and then at reflux for 6 h. After cooling, the suspension is filtered off, washed with glacial acetic acid, methanol and hot water (about 90° C.) and dried at 60° C. The yield is 4.42 g (80% of theory) of a red powder of the following formula:

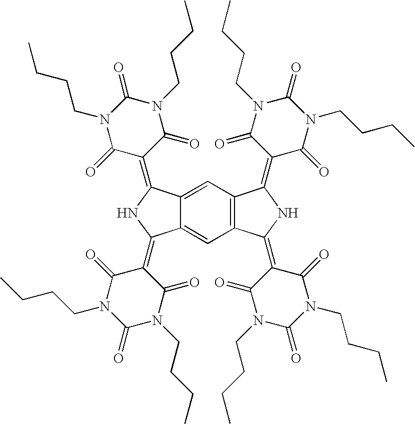

Melting point: >300° C.

MS (m/e): 1106 [M+H]⁺

### Example 5

### 5,5′,5″,5′″-Benzo[1,2-c:4,5-c′]dipyrrole-1,3,5,7(2H,6H)-tetraylidenetetrakis-1,3-didodecyl-2,4,6-trioxotetrahydropyrimidine

1.06 g of benzo[1,2-c:4,5-c′]dipyrrole-1,3,5,7(2H,6H)-tetraimine and 11.6 g of 1,3-didodecylbarbituric acid are stirred in a mixture of 20 ml of glacial acetic acid and 80 ml of toluene at room temperature for 24 h and then at reflux for 6 h. After cooling, the suspension is filtered off, washed with glacial acetic acid, methanol and hot water (about 90° C.) and dried at 60° C. Recrystallization may be effected from chloroform-methanol mixtures. The yield is 3.41 g (34% of theory) of a red powder of the following formula:

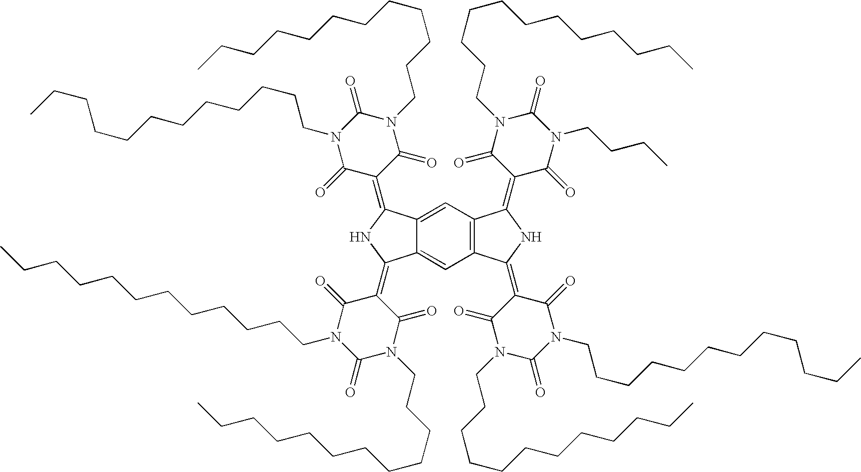

Melting point: 207° C.

MS (m/e): 2003 [M−H]⁻

### Example 6

### 2,2′,2″,2′″-Benzo[1,2-c:4,5-c′]dipyrrole-1,3,5,7(2H,6H)-tetraylidenetetrakis-1,3-dioxoindane

3.88 g of benzo[1,2-c:4,5-c′]dipyrrole-1,3,5,7(2H,6H)-tetraimine and 16.1 g of 1,3-dioxoindane are placed in 110 ml of glacial acetic acid and first stirred at room temperature for 24 h and then heated at the boil for 6 h. The suspension is cooled down to room temperature, filtered off, washed in succession with glacial acetic acid, methanol and hot water (about 90° C.) and subsequently dried at 60° C. The yield is 12.9 g (97% of theory) of a brownish red powder of the following formula:

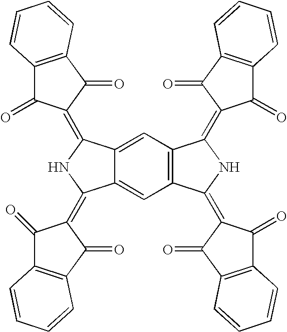

Melting point: >300° C.

MS (m/e): 728 [M−H]⁻

### Example 7

### Reaction of benzo[1,2-c:4,5-c′]dipyrrole-1,3,5,7(2H,6H)-tetraimine with aniline and 2,4-dihydroxyquinoline

5.3 g of benzo[1,2-c:4,5-c′]dipyrrole-1,3,5,7(2H,6H)-tetraimine and 8.1 g of 2,4-dihydroxyquinoline are stirred in 150 ml of glacial acetic acid at room temperature for 24 hours. 7.0 g of aniline are added before heating at the boil for 6 hours. The suspension is cooled down to room temperature, filtered off, washed in succession with glacial acetic acid, methanol and hot water (about 90° C.) and subsequently dried at 60° C. The yield is 11.0 g of a brownish red powder of a mixture of the following formulae:

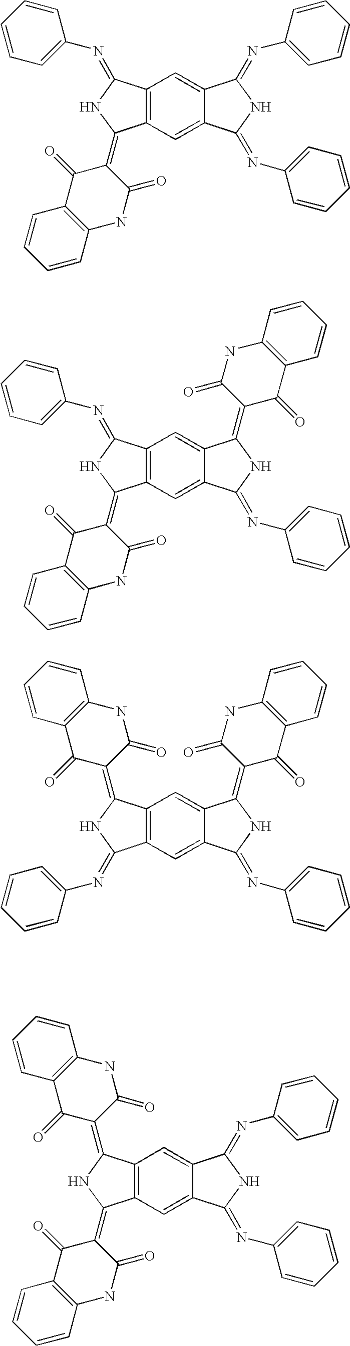

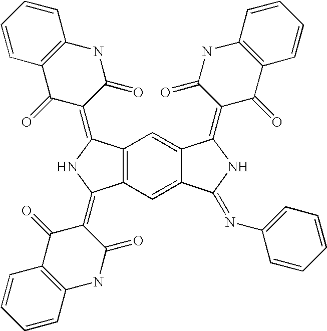

Melting point: >300° C.

MALDI (m/e): 586, 654, 722 [M+H]⁺

### Example 8

### 5,5′,5″,5′″-Benzo[1,2-c:4,5-c′]dipyrrole-1,3,5,7(2H,6H)-tetraylidenetetrakis-1,3-diphenyl-2,4,6-trioxotetrahydropyrimidine

1.06 g of benzo[1,2-c:4,5-c′]dipyrrole-1,3,5,7(2H,6H)-tetraimine and 8.41 g of 1,3-diphenylbarbituric acid are stirred in a mixture of 10 ml of glacial acetic acid and 40 ml of toluene at 50° C. for 1 h and then at reflux for 6 h. After cooling, the suspension is filtered off, washed with glacial acetic acid, methanol and hot water (about 90° C.) and dried at 60° C. The yield is 5.21 g (82% of theory) of a red powder of the following formula

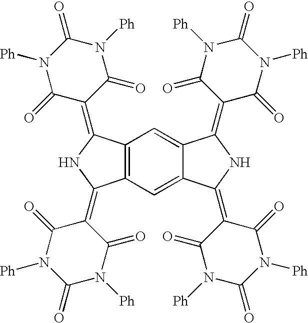

Melting point: >300° C.

MS (m/e): 1266 [M+H]⁺, 1288 [M+Na]⁺

### Example 9

### 5,5′,5″,5′″-Benzo[1,2-c:4,5-c′]dipyrrole-1,3,5,7(2H,6H)-tetraylidenetetrakis-2,4,6-trioxotetrahydropyrimidine

A suspension of 11.3 g of 1,2,4,5-tetracyanobenzene and 3.0 g of sodium methoxide solution (30% in methanol) in 340 ml of methanol is stirred at room temperature for 16 hours. 6.8 g of glacial acetic acid and 34.0 g of barbituric acid are added and the reaction mixture is subsequently heated at room temperature for 24 hours and then at the boil for 6 hours. The suspension is cooled down to room temperature and filtered. The crude product is purified by stirring in 250 ml of glacial acetic acid at the boil for 15 min, hot filtration with suction, successive washing with glacial acetic acid and hot water (about 90° C.) and subsequent drying at 60° C. The yield is 38.2 g (92% of theory) of a brown powder of the following formula:

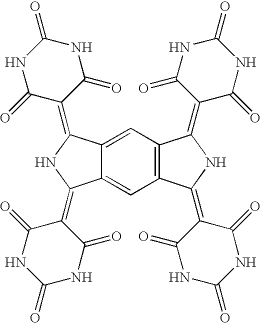

Melting point: >300° C.

MALDI (m/e): 656 [M−H]⁻

### Application Examples

To assess the properties of the pigments prepared in accordance with the invention in the coatings sector a selection was made, from among the multiplicity of known varnishes, of an aromatics-containing alkyd-melamine resin varnish (AM) based on a medium-oil alkyd resin and on a butanol-etherified melamine resin, and also of an aromatics-free, air-drying alkyd resin varnish (LA) based on a long-oil soya alkyd resin.

To assess the properties of the polymer-soluble dyes of the invention, crystal grade polystyrene was selected as plastic for coloring. Test specimens were produced by injection molding.

### Application Example 1

Application of the pigment from example 1a in AM varnish produces a clean bluish red coating in the masstone and a strong reddish orange coating in the white reduction.

### Application Example 2

Application of the pigment from example 1b in AM varnish produces a clean bluish red coating in the masstone and a strong reddish orange coating in the white reduction.

### Application Example 3

Application of the pigment from example 2 in AM varnish produces strong yellowish orange coatings in the masstone and in the white reduction.

### Application Example 4

Application of the pigment from example 3 in LA varnish produces yellowish orange coatings in the masstone and in the white reduction.

### Application Example 5

Application of the pigment from example 3 in polystyrene produces strong reddish orange specimens in the masstone and in the white reduction.

### Application Example 6

Application of the pigment from example 4 in AM varnish produces a clean red coating in the masstone and a strong neutral red coating in the white reduction.

### Application Example 7

Application of the pigment from example 4 in polystyrene produces clean and strong reddish orange specimens in the masstone and in the white reduction.

### Application Example 8

Application of the pigment from example 5 in polystyrene produces clean and strong reddish orange specimens in the masstone and in the white reduction.

### Application Example 9

Application of the pigment from example 6 in AM varnish produces a hiding yellowish red coating in the masstone and a strong reddish brown coating in the white reduction.

### Application Example 10

Application of the pigment from example 7 in AM varnish produces a transparent brown coating in the masstone and a yellowish brown coating in the white reduction.

### Application Example 11

Application of the pigment from example 8 in AM varnish produces a hiding bordeaux coating in the masstone and a strong bluish red coating in the white reduction.

### Application Example 12

Application of the pigment from example 9 in AM varnish produces a hiding brown coating in the masstone and a neutral brown coating in the white reduction.

1) A compound of the general formula (I) 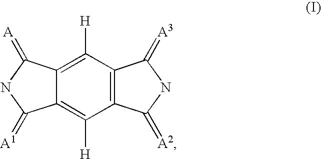
wherein A is a divalent radical of a cyclic compound of the general formula (II) 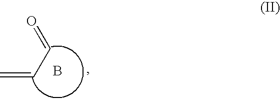
wherein B is an alicyclic or heterocyclic group, and
A¹, A² and A³ are the same or different and are A or ═NR, wherein R is hydrogen, unsubstituted phenyl, phenyl substituted by 1, 2, 3 or 4 radicals selected from the group consisting of halogen, R⁰, OR⁰, SR⁰, NH₂, NHR⁰, NR⁰₂, NO₂, COOH, COOR⁰, CONH₂, CONHR⁰, CONR⁰₂, CN, SO₃H, SO₂(OR⁰), and SO₂R⁰, phenyl substituted by a 5- to 7-membered heteroaromatic radical having 1, 2 or 3 heteroatoms selected from the group consisting of N, O and S; a 5- to 7-membered heteroaromatic radical having 1, 2 or 3 heteroatoms selected from the group consisting of N, O and S; NH₂, NHR⁰, NR⁰₂, NHCONH₂ or NHCONHR⁰, where R⁰ is C₁-C₁₈-alkyl or C₆-C₂₄-aryl.

2) The compound according to claim 1 wherein one of A¹, A² and A³ is A.

3) The compound according to claim 1 wherein two of A¹, A² and A³ are A.

4) The compound according to claim 1 wherein A¹, A² and A³ are A.

5) The compound according to claim 1, wherein A is a divalent radical of a cyclic compound of the general formula (III) 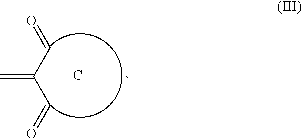
wherein C is an alicyclic or heterocyclic group.

6) The compound according to claim 1, wherein A is a divalent radical of the formulae (a) to (g) 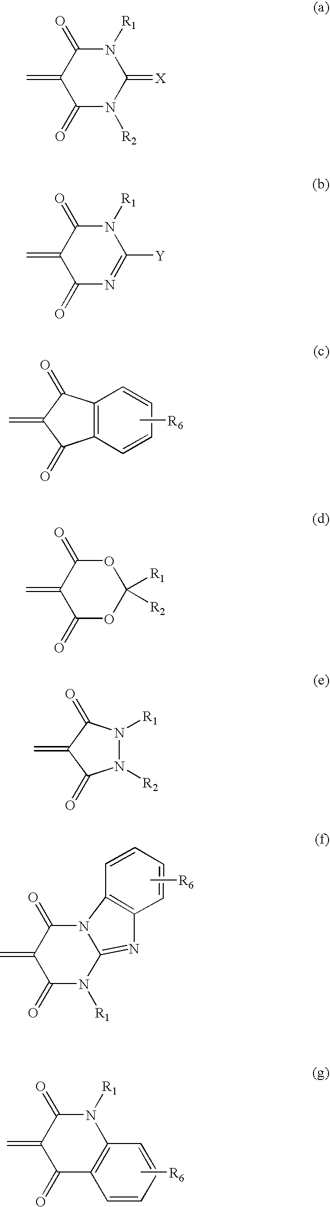
wherein R₁ and R₂ independently represent hydrogen, C₁-C₂₅-alkyl, C₅-C₁₂-cycloalkyl, C₆-C₂₄-aryl, C₁-C₂₅-alkyl-(C₆-C₁₀-aryl), a 5- to 7-membered heteroaromatic radical having 1, 2 or 3 heteroatoms selected from the group consisting of N, O and S, —(CH₂)ₙ—COR₃ or —(CH₂)ₘ—OR₄,
wherein R₃ is hydroxyl, amino or unsubstituted or at least singularly hydroxyl- or amino-substituted C₁-C₂₅-alkoxy, C₁-C₂₅-alkylamino, di(C₁-C₂₅-alkyl)amino, C₆-C₂₄-arylamino, di(C₆-C₂₄-aryl)amino, C₁-C₂₅-alkyl-(C₆-C₁₀-aryl)amino or C₂-C₂₄-alkenyloxy;
R₄ is hydrogen or —CO—(C₁-C₂₅-alkyl), and
n and m are independently an integer from 0 to 6,
and wherein a C—C unit in R₁, R₂, R₃ and R₄ is, optionally, replaced by an ether unit C—O—C;
X is ═O, ═S or ═NR₅, where R₅ is R₁ or R₂;
Y is hydrogen, R₂, OR₂, SR₂, NHCN or NR₂R₅;
and R₆ is hydrogen, halogen, CN, R₂, OR₂, SR₂, NR₂R₅, NO₂, SO₂(OR₂), SO₂R₂, SO₂NR₂R₅ or PO₂(OR₂).

7) The compound according to claim 6 wherein
R₁ and R₂ are hydrogen, C₁-C₁₈-alkyl, C₅-C₆-cycloalkyl, C₆-C₁₀-aryl, benzyl, pyridyl, pyrryl, thienyl, imidazolyl, oxazolyl, thiazolyl, pyrimidyl, hydroxycarbonyl-C₀-C₆-alkyl, C₁-C₁₈-alkoxycarbonyl-C₀-C₆-alkyl, aminocarbonyl-C₀-C₆-alkyl, C₁-C₁₈-alkylaminocarbonyl-C₀-C₆-alkyl, C₆-C₁₀-arylaminocarbonyl-C₀-C₆-alkyl, di(C₁-C₁₈-alkyl)aminocarbonyl-C₀-C₆-alkyl, C₁-C₁₈-alkyl-C₆-C₁₀-arylaminocarbonyl-C₀-C₆-alkyl or di(C₆-C₁₀-aryl)aminocarbonyl-C₀-C₆-alkyl.

8) The compound according to claim 6 wherein R₃ is hydroxyl, C₁-C₁₈-alkoxy, C₁-C₁₈-alkylamino, di(C₁-C₁₈-alkyl)amino, benzylamino, C₆-C₁₀-arylamino, di(C₆-C₁₀-aryl)amino or (C₂-C₁₈)-alkenyloxy.

9) The compound according to claim 6, wherein
R₆ is hydrogen, Cl, Br, C₁-C₁₈-alkyl, C₅-C₆-cycloalkyl, benzyl, C₆-C₁₀-aryl, pyridyl, pyrryl, thienyl, imidazolyl, oxazolyl, thiazolyl, pyrimidyl, C₁-C₁₈-alkoxy, C₆-C₁₀-aryloxy, C₁-C₁₈-alkylthio, C₆-C₁₀-arylthio, C₁-C₁₈-alkylamino, C₆-C₁₀-arylamino, di(C₁-C₁₈-alkyl)amino, C₁-C₁₈-alkyl-C₆-C₁₀-arylamino, di(C₆-C₁₀-aryl)amino, SO₃H, C₁-C₁₈-alkoxysulfonyl, C₁-C₁₈-alkylsulfonyl or di(C₁-C₁₈-alkyl)aminosulfonyl.

10) A process for preparing a compound according to claim 1, comprising the steps of reacting 1,2,4,5-tetracyanobenzene with at least 2 equivalents of at least one of ammonia or alkoxides MOR₇,
wherein M is sodium or potassium, R₇ is C₁-C₁₈-alkyl or —(CH₂)ₘ—OH and m is an integer from 1 to 6, and a C—C unit is, optionally, replaced by an ether unit C—O—C, in a solvent or solvent mixture under basic to neutral conditions at a temperature in the range from −20 to 120° C. to form tetra-, tri-, di- or monoimino-substituted benzodipyrroles, and
reacting the tetra-, tri-, di- or monoimino-substituted benzodipyrroles with at least 1 equivalent of a cyclic compound of the formula (VII) in a solvent or solvent mixture under neutral to acidic conditions 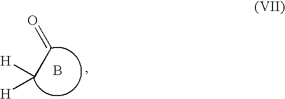
and optionally not more than 3 equivalents of H₂NR.

11) A method of using a compound according to claim 1 comprising the step of dyeing or pigmenting an organic or inorganic, high or low molecular weight material with the compound.

12) The method according to claim 11 wherein the material is selected from the group consisting of electrophotographic toners, electrophotographic developers, color filters, electronic inks and optical layers for optical data storage.

13) A composition comprising an organic or inorganic, high or low molecular weight material and at least one of the compound as claimed in claim 1, in an amount from 0.005% to 70% by weight, based on the material.

14) A composition comprising an organic or inorganic, high or low molecular weight material and at least one of the compound as claimed in claim 1.

15) The composition as claimed in claim 14, wherein the composition is an electrophotographic toner, electrophotographic developer, color filter, electronic ink or an optical layer for optical data storage.
